(19) United States
(12) Patent Application Publication
(10) Pub. No.: US 2010/0056574 A1
(43) Pub. Date: Mar. 4, 2010

(54) Crystalline Forms of Sufentanil

(75) Inventors: Brian K. Cheng, Chesterfield, MO (US); Gary A. Nichols, Wildwood, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/546,898

(22) Filed: Aug. 25, 2009

Related U.S. Application Data
(60) Provisional application No. 61/094,087, filed on Sep. 4, 2008.
(60) Provisional application No. 61/093,829.

(51) Int. Cl. A61K 31/4535; C07D 409/06; A61P 25/00

(52) U.S. Cl. 514/326; 546/213

(57) ABSTRACT

The present invention provides crystalline forms of sufentanil citrate and methods for preparing crystalline forms of sufentanil citrate.

### CROSS REFERENCE TO RELATED APPLICATIONS

This application claims the benefit of the filing date of U.S. Provisional Application Ser. No. 61/094,087, filed Sep. 4, 2008, entitled “Crystalline Forms of Sufentanil” which is incorporated herein in their entirety.

### FIELD OF THE INVENTION

The present invention generally relates to crystalline forms of sufentanil and processes for preparing crystalline forms of sufentanil. In particular, the present invention relates to crystalline forms and processes for preparing crystalline forms of the citrate salt of sufentanil.

### BACKGROUND OF THE INVENTION

Solids exist in either amorphous or crystalline forms. In the case of crystalline forms, molecules are positioned in three-dimensional lattice sites. When a compound recrystallizes from a solution or slurry, it may crystallize with different spatial lattice arrangements, and the different crystalline forms are sometimes referred to as “polymorphs.” The different crystalline formic forms of a given substance may differ from each other with respect to one or more chemical properties (e.g., dissolution rate, solubility), biological properties (e.g., bioavailability, pharmacokinetics), and/or physical properties (e.g., mechanical strength, compaction behavior, flow properties, particle size, shape, melting point, degree of hydration or salvation, caking tendency, compatibility with excipients). The variation in properties among different crystalline forms usually means that one crystalline form is desired or preferred over other forms.

Sufentanil is a member of the series of potent fentanyl analogues. It is characterized by a high selectivity and affinity (approximately 10 times greater than fentanyl) for “mu” opiate receptors. When compared with fentanyl, sufentanil's pharmacokinetic profile shows a smaller volume of distribution, resulting in a terminal half-life intermediate between alfentanil and fentanyl. Additionally, sufentanil, like fentanyl, does not cause histamine release. The chemical name for sufentanil is: N-[4-(methoxymethyl)-1-[2-(2-thienyl)ethyl]-4-piperidinyl]-N-phenylpropanamide. In its citrate form, the chemical name is N-[4-(methoxymethyl)-1-[2-(2-thienyl)ethyl]-4-piperidinyl]-N-phenylpropanamide, 2-hydroxy-1,2,3-propanetricarboxylate. Synthesis of sufentanil was first disclosed in U.S. Pat. No. 3,998,834 to Janssen. An improved method of synthesis is described in U.S. Pat. No. 5,489,689 to Mallinckrodt, which describes a shorter method for the production of sufentanil, which can subsequently be converted to the hydrochloride salt or citrate salt. Subsequently, a further-improved method for the synthesis of sufentanil is disclosed in U.S. Pat. No. 7,208,604 to Matthew. No crystalline forms of sufentanil, however, have been characterized.

Because sufentanil exhibits most of the properties of an ideal analgesic, improved forms of the compound are desired, particularly with regard to enhanced solubility, bioavailability, ease of synthesis, ability to be readily formulated, and/or physical stability. Furthermore, there is a need for methods to produce improved crystalline forms of sufentanil.

### SUMMARY OF THE INVENTION

The present invention provides crystalline forms of sufentanil citrate and processes for producing crystalline forms of sufentanil citrate. Among the various aspects of the invention is a provision for crystalline Form II of sufentanil citrate, N-[4-(methoxymethyl)-1-[2-(2-thienyl)ethyl]-4-piperidinyl]-N-phenylpropanamide, 2-hydroxy-1,2,3-propanetricarboxylate.

Another aspect of the invention encompasses a pharmaceutical composition comprising crystalline Form II of sufentanil citrate, N-[4-(methoxymethyl)-1-[2-(2-thienyl)ethyl]-4-piperidinyl]-N-phenylpropanamide, 2-hydroxy-1,2,3-propanetricarboxylate, and a pharmaceutically acceptable excipient.

A further aspect of the invention provides a first process for preparing a substantially pure anhydrous crystalline Form I of sufentanil citrate, N-[4-(methoxymethyl)-1-[2-(2-thienyl)ethyl]-4-piperidinyl]-N-phenylpropanamide, 2-hydroxy-1,2,3-propanetricarboxylate. The process comprises contacting sufentanil citrate, N-[4-(methoxymethyl)-1-[2-(2-thienyl)ethyl]4-piperidinyl]-N-phenylpropanamide, 2-hydroxy-1,2,3-propanetricarboxylate, with a solvent to form a saturated or a near saturated solution, and forming crystals of substantially pure crystalline Form I.

Yet another aspect of the invention encompasses a process for preparing a substantially pure hydrous crystalline Form II of sufentanil citrate, N-[4-(methoxymethyl)-1-[2-(2-thienyl)ethyl]-4-piperidinyl]-N-phenylpropanamide, 2-hydroxy-1,2,3-propanetricarboxylate. The process comprises contacting sufentanil citrate, N-[4-(methoxymethyl)-1-[2-(2-thienyl)ethyl]-4-piperidinyl]-N-phenylpropanamide, 2-hydroxy-1,2,3-propanetricarboxylate, with a solvent to form a saturated a near saturated solution, and forming crystals of substantially pure crystalline Form II.

Other aspects and features of the invention will be in part apparent and in part described in more detail below.

### DESCRIPTION OF THE FIGURES

FIG. 1 represents an X-ray powder diffraction pattern of crystalline Form I of sufentanil citrate. Peak intensity is plotted as a function of degrees 2-theta.

FIG. 2 represents a differential scanning calorimetry (DCS) thermogram of crystalline Form I of sufentanil citrate. Heat flux is plotted as a function of temperature.

FIG. 3 represents an X-ray powder diffraction pattern of crystalline Form II of sufentanil citrate. Peak intensity is plotted as a function of degrees 2-theta.

FIG. 4 represents a differential scanning calorimetry (DCS) thermogram of crystalline Form II of sufentanil citrate. Heat flux is plotted as a function of temperature.

### DETAILED DESCRIPTION

The present invention provides two crystalline forms (i.e., Form I and Form II) of the citrate salt of sufentanil. Each crystalline form exhibits a characteristic profile of X-ray powder diffraction peaks and exhibits a characteristic melting endotherm as measured by differential scanning calorimetry. The invention also provides a pharmaceutical composition comprising crystalline Form II of sufentanil citrate and a pharmaceutically acceptable excipient. Also provided are processes for producing the two crystalline forms of sufentanil citrate.

### (I) Crystalline Forms of Sufentanil Citrate

A first aspect of the invention encompasses crystalline forms of the citrate salt of sufentanil. It has been discovered that crystalline sufentanil citrate exists in different crystalline forms. Anhydrous Form I is the predominate form in sufentanil citrate produced by Mallinckrodt Inc. (St. Louis, Mo.) and is characterized herein. Hydrous Form II of sufentanil citrate is also characterized herein. The two crystalline forms may be distinguished on the basis of different X-ray powder diffraction patterns. The two crystalline forms also may be distinguished on the basis of different endothermic transitions, as determined by differential scanning calorimetry. Those of skill in the art will appreciate that other analytical techniques, such as single crystal X-ray diffraction analysis, Fourier transform infrared spectroscopy, etc., also may be used to distinguish the two crystalline forms.

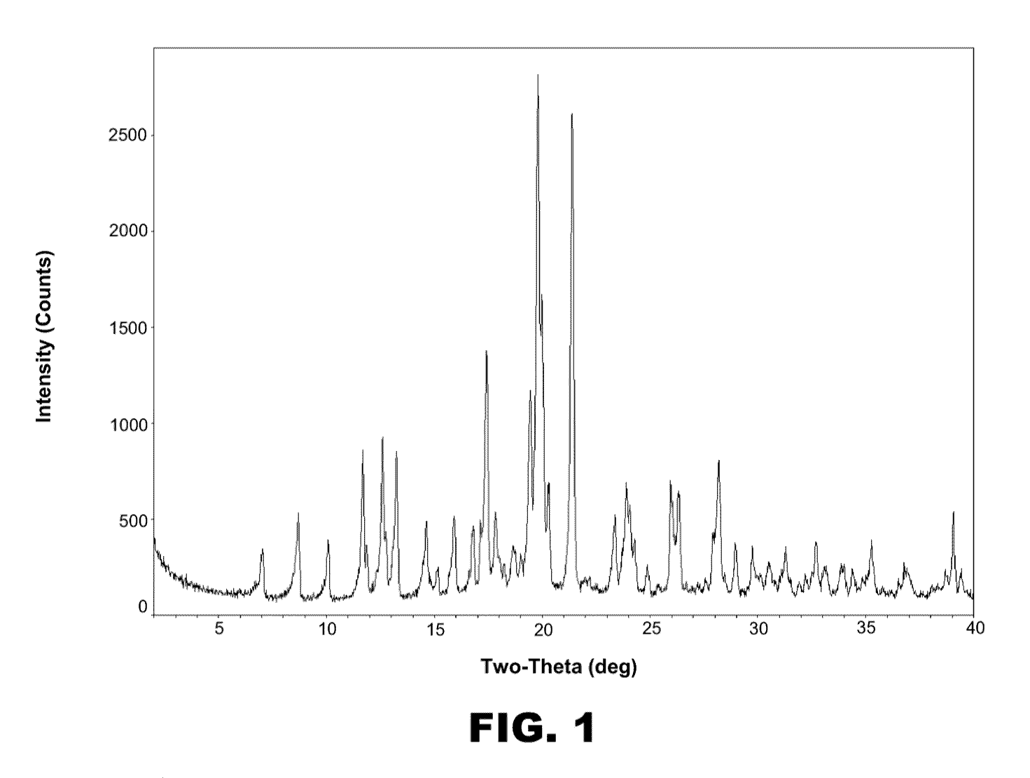

Crystalline sufentanil citrate may exist as anhydrous crystalline Form I. Crystalline Form I of sufentanil citrate exhibits an X-ray powder diffraction pattern comprising characteristic peaks expressed in degrees 2-theta as diagrammed in FIG. 1. In particular, Form I exhibits characteristic peaks expressed in degrees 2-theta at about 11.7, about 12.6, about 13.3, about 17.4, about 19.5, about 19.8, and about 21.4. In general, the peak with the highest intensity is at about 19.8 degrees 2-theta, and the peak with the second highest intensity is at about 21.4 degrees 2-theta.

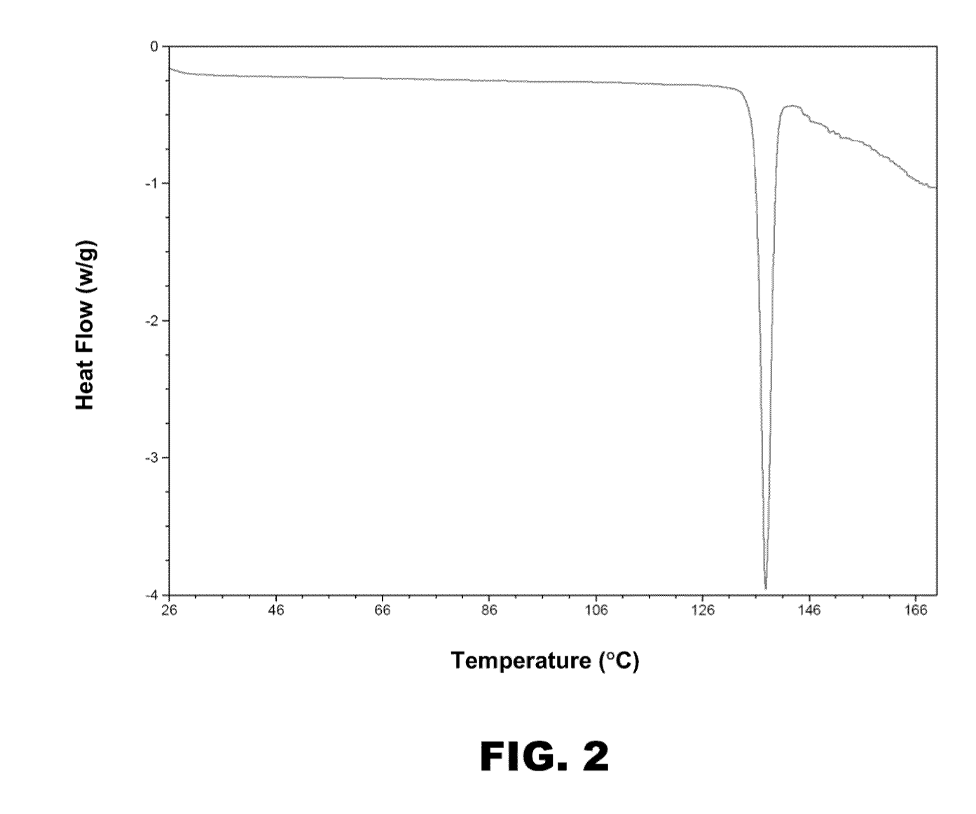

Form I of sufentanil citrate exhibits a characteristic melting endoderm, as depicted in the differential scanning calorimetry thermogram shown in FIG. 2. In particular, crystalline Form I exhibits an endothermic transition with an onset of about 136-138° C. as measured by differential scanning calorimetry (at a scan rate of 5° C. per minute).

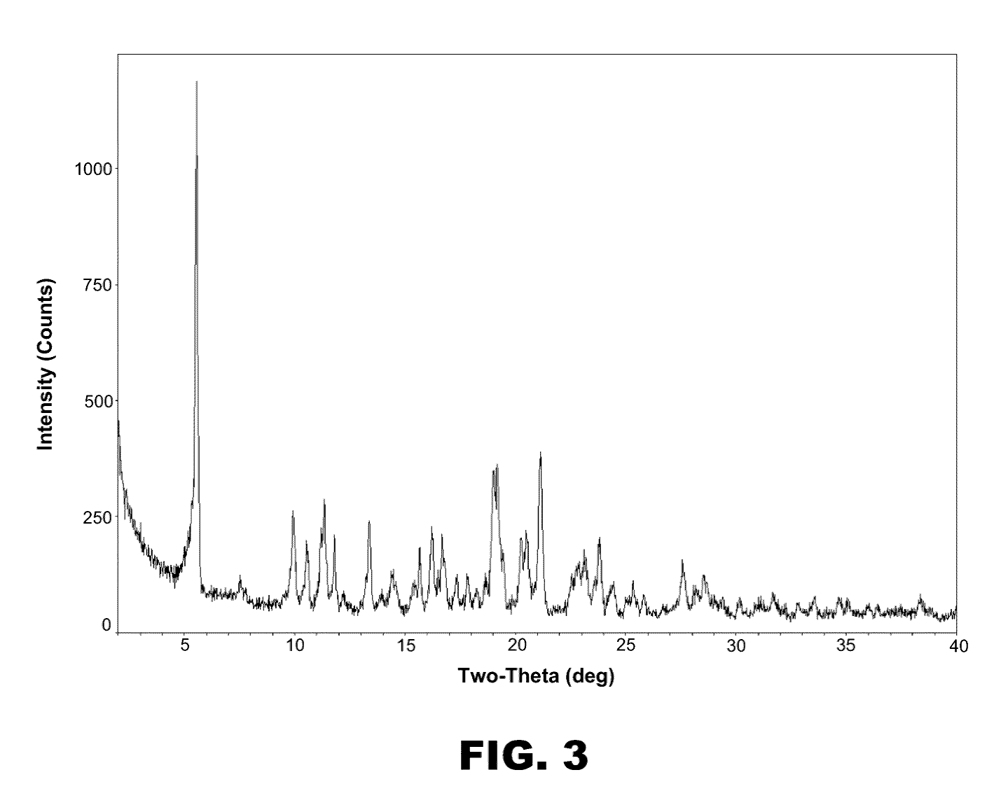

Crystalline sufentanil citrate may also exist as hydrous crystalline Form II. This crystalline form exhibits an X-ray powder diffraction pattern comprising characteristic peaks expressed in degrees 2-theta as diagrammed in FIG. 3. In particular, Form II exhibits characteristic peaks expressed in degrees 2-theta at about 5.6, about 10.0, about 11.4, about 13.4, about 19.1, and about 21.2. In general, the peak with the highest intensity is at about 5.6 degrees 2-theta. Peaks with the next highest intensities may be at about 19.1 and 21.2 degrees 2-theta.

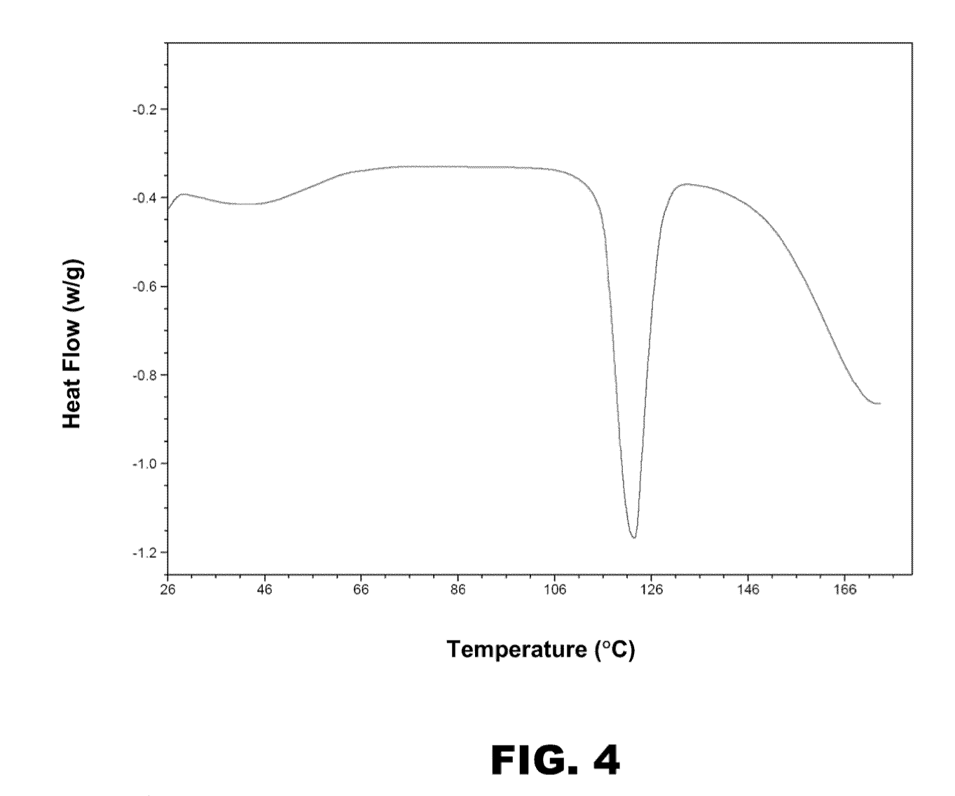

Form II of sufentanil citrate also exhibits a characteristic melting endoderm, as depicted in the differential scanning calorimetry thermogram shown in FIG. 4. In particular, crystalline Form II exhibits a broad endothermic transition below about 75° C. and an endothermic transition with an onset of about 114-118° C. as measured by differential scanning calorimetry (at a scan rate of 5° C. per minute).

In general, each of the crystalline forms of sufentanil citrate is substantially pure. The phrase “substantially pure,” as used herein, means that the crystalline form has a purity of about 95%, or more preferably about 97%, by weight as defined by X-ray powder diffraction. Stated another way, the crystalline form has no more than about 5% by weight, or more preferably no more than about 3% by weight, of another crystalline form of sutentanil citrate.

### (II) Pharmaceutical Composition

Another aspect of the invention provides for a pharmaceutical composition comprising crystalline Form II of sufentanil citrate and a pharmaceutically acceptable excipient. In general, the sufentanil citrate of the pharmaceutical composition will comprise about 95% of Form II by weight, and more preferably about 97% of Form II by weight, and no more than about 5% by weight of Form I, and preferably no more than about 3% by weight of Form I. Crystalline Form II of sufentanil citrate is characterized above in section (I).

A variety of excipients commonly used in pharmaceutical formulations may be selected on the basis of several criteria such as, e.g., the desired dosage form and the release profile properties of the dosage form. Non-limiting examples of suitable excipients include an agent selected from the group comprising a binder, a filler, a non-effervescent disintegrant, an effervescent disintegrant, a preservative, a diluent, a flavoring agent, a sweetener, a lubricant, an oral dispersing agent, a coloring agent, a taste masking agent, a pH modifier, a stabilizer, a compaction agent, and combinations of any of these agents.

In one embodiment, the excipient may be a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, peptides, and combinations thereof.

In another embodiment, the excipient may be a filler. Suitable fillers include carbohydrates, inorganic compounds, and polyvinylpirrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may be a non-effervescent disintegrant. Suitable examples of non-effervescent disintegrants include starches (such as corn starch, potato starch, and the like), pregelatinized and modified starches thereof, sweeteners, clays (such as bentonite), micro-crystalline cellulose, alginates, sodium starch glycolate, and gums (such as agar, guar, locust bean, karaya, pecitin, and tragacanth).

In another embodiment, the excipient may be an effervescent disintegrant. By way of non-limiting example, suitable effervescent disintegrants include sodium bicarbonate in combination with citric acid, and sodium bicarbonate in combination with tartaric acid.

The excipient may comprise a preservative. Suitable examples of preservatives include antioxidants (such as alpha-tocopherol or ascorbate) and antimicrobials (such as parabens, chlorobutanol or phenol). In other embodiments, an antioxidant such as butylated hydroxytoluene (BHT) or butylated hydroxyanisole (BHA) may be utilized.

In another embodiment, the excipient may include a diluent. Diluents suitable for use include pharmaceutically acceptable saccharides such as sucrose, dextrose, lactose, microcrystalline cellulose, fructose, xylitol, and sorbitol; polyhydric alcohols; starches; pre-manufactured direct compression diluents; and mixtures of any of the foregoing.

The excipient may include flavoring agents. Flavoring agents may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot).

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose.

Depending upon the embodiment, it may be desirable to provide a coloring agent. Suitable color additives include food, drug and cosmetic colors (FD&C), drug and cosmetic colors (D&C), or external drug and cosmetic colors (Ext. D&G). These colors or dyes, along with their corresponding lakes, and certain natural and derived colorants may be suitable for use in the present invention depending on the embodiment.

The excipient may include a taste-masking agent. Taste-masking materials include cellulose hydroxypropyl ethers (HPC); low-substituted hydroxypropyl ethers (L-HPC); cellulose hydroxypropyl methyl ethers (HPMC); methylcellulose polymers and mixtures thereof; polyvinyl alcohol (PVA); hydroxyethylcelluloses; carboxymethylcelluloses and salts thereof; polyvinyl alcohol and polyethylene glycol co-polymers; monoglycerides or triglycerides; polyethylene glycols; acrylic polymers; mixtures of acrylic polymers with cellulose ethers; cellulose acetate phthalate; and combinations thereof.

In various embodiments, the excipient may include a pH modifier. In certain embodiments, the pH modifier may include sodium carbonate or sodium bicarbonate.

The weight fraction of the excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

The pharmaceutical compositions detailed herein may be manufactured in one or several dosage forms. Suitable dosage forms include tablets, including suspension tablets, chewable tablets, effervescent tablets or caplets; pills; powders such as a sterile packaged powder, a dispensable powder, and an effervescent powder; capsules including both soft or hard gelatin capsules such as HPMC capsules; lozenges; a sachet; a sprinkle; a reconstitutable powder or shake; a troche; pellets such as sublingual or buccal pellets; granules; liquids for oral or parenteral administration; suspensions; emulsions; semisolids; or gels.

The dosage forms may be manufactured using conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., priling, spray drying, pan coating, melt granulation, granulation, wurster coating, tangential coating, top spraying, extruding, coacervation and the like.

In general, the pharmaceutical compositions of the invention will be used for analgesia and anesthesia, most often in operating rooms or intensive care units. While the pharmaceutical compositions typically will be used for surgical procedures of short duration or in situations where a rapid suppression of reflex responses is required, they may also be used for operations of longer duration. In this case, the composition may be bolus administered followed by infusion at a rate sufficient to compensate for the disappearance of the active ingredient due to redistribution and elimination.

The amount of active ingredient that is administered to a subject can and will vary depending upon a variety of factors such as the age and overall health of the subject, and the particular mode of administration. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference.

### (III) Processes for Preparing Crystalline Forms of Sufentanil Citrate

Another aspect of the present invention provides processes for producing substantially pure crystalline forms of sufentanil citrate. In particular, a process is provided for the preparation of the anhydrous Form I crystalline form, and a process is provided for the preparation of the hydrous Form II crystalline form.

(a) Process for Preparing Crystalline Form I

The process for preparing Form I comprises (a) contacting sufentanil citrate with a solvent to form a saturated or near saturated solution of sufentanil citrate, and (b) forming crystals of substantially pure crystalline Form I. The sufentanil citrate that is contacted with the solvent may be in a solid form (e.g., a powder) or a liquid form (e.g., in a solution comprising a co-solvent, or a concentrated oil/gel/gum). The solvent used in the process can and will vary depending upon the embodiment. The solvent may be a protic solvent, an aprotic solvent, or a combination thereof. Suitable protic solvents include, but are not limited to, methanol, ethanol, isopropanol, n-propanol, isobutanol, n-butanol, s-butanol, t-butanol, formic acid, acetic acid, or combinations thereof. Non-limiting examples of suitable aprotic solvents include acetone, acetonitrile, dichloromethane, tetrahydrofuran, or combinations thereof. In a preferred embodiment, the solvent may be an alcohol such as methanol, ethanol, isopropanol, n-propanol, isobutanol, n-butanol, s-butanol, or t-butanol. In an exemplary embodiment, the solvent may be ethanol or isopropanol. The weight ratio of solvent to sufentanil citrate may range from about 5:1 to about 20:1, or more preferably from about 5:1 to about 10:1.

The temperature of the process can and will vary. The temperature of step (a) may range from about 4° C. to about the boiling temperature of the solvent. In a preferred embodiment, step (a) may be conducted at about room temperature. Step (b) of the process may be conducted at a temperature that ranges from about −10° C. to about 40° C., or more preferably from about 0° C. to about 25° C.

The crystals of anhydrous Form I may be formed by one of several different methods. In one embodiment, the crystals may be formed by “slow evaporation crystallization.” For this, the solvent is typically slowly evaporated such that crystals slowly form. The rate of evaporation may be slowed by placing the saturated or near saturated solution in a flask with a narrow opening, covering the opening with paper or foil comprising a few small holes, or sealing the opening with a cap into which a needle has been inserted. Evaporation of the solvent may be conducted at atmosphere or in an inert environment (i.e., under nitrogen or argon). The solvent may be evaporated at atmospheric pressure or at a pressure that is less than atmospheric pressure. In a preferred embodiment, the solvent is evaporated at atmospheric pressure in an inert environment at room temperature.

In another embodiment, Form I crystals may be formed by “hot crystallization.” In this embodiment, step (a) is conducted at an elevated temperature. Typically, sufentanil citrate is contacted with the solvent at a temperature that is at or near the boiling point of the solvent. Crystals are formed in step (b) by cooling the saturated solution of sufentanil citrate to a temperature that ranges from about −10° C. to about 40° C., or more preferably from about 0° C. to about 25° C.

The process generally further comprises collecting the crystals of substantially pure crystalline Form I. The crystals may be collected by filtration, centrifugation, or other techniques well known in the art. The process may further comprise drying the crystals of substantially pure crystalline Form I. The crystals may be dried under a vacuum either at room temperature or at an elevated temperature.

(b) Process for Preparing Crystalline Form II

The process for preparing Form II comprises (a) contacting sufentanil citrate with a solvent to form a saturated or near saturated solution of sufentanil citrate, and (b) forming crystals of substantially pure crystalline Form II. The sufentanil citrate that is contacted with the solvent may be in a solid form (e.g., a powder) or a liquid form (e.g., in a solution comprising a co-solvent, or a concentrated oil/gel/gum). The solvent may be a protic solvent, an aprotic solvent, or a combination thereof. Suitable solvents are presented above; suitable protic solvents also include water. In a preferred embodiment, the solvent may be water.

The process for producing crystalline Form II of sufentanil citrate generally utilizes “hot crystallization” as detailed above. The method generally further comprises collecting the crystals, as detailed above. The process may further comprise drying the crystals of substantially pure crystalline Form II, as detailed above.

### EXAMPLES

The following examples illustrate various embodiments of the invention.

### Example 1

### Preparation and Characterization of Form I Utilizing Slow Evaporation Crystallization

Sufentanil citrate was mixed with ethanol to form a saturated or near saturated solution of sufentanil citrate. The solution was transferred to a small vial and sealed with a septa-cap. A needle was poked through the septa-cap and the vial was placed in a room temperature nitrogen-purged dessicator. The needle allowed for slow evaporation and slow crystal growth. The crystals were collected and dried to obtain substantially pure Form I.

The crystalline Form I was characterized by X-ray powder diffraction spectrometry. The diffraction pattern was obtained using a Bruker/Siemens D500 X-ray diffractometer, equipped with a graphite monochromator, and a Cu X-ray source operated at 40 kV, 30 mA, over the range of 2-40 degrees 2-theta. Table 1 summarizes the X-ray powder diffraction data, i.e., 2-theta degree positions of the peaks, height of the peaks, area of the peaks, and so forth. FIG. 1 presents the characteristic X-ray powder diffraction pattern for the crystalline Form I. This crystalline form exhibited characteristic peaks at about 11.7, about 12.6, about 13.3, about 17.4, about 19.5, about 19.8, and about 21.4 degrees 2-theta.

TABLE 1
X-Ray Powder Diffraction Spectral Lines of Form I.

| | d Value | | Intensity | | | | |
|---|---|---|---|---|---|---|---|
| 2-Theta | (Å) | Background | Height | % | Intensity Area | % | FWHM* |
| 7.061 | 12.5087 | 96 | 245 | 9.3 | 3033 | 7.2 | 0.210 |
| 8.722 | 10.1299 | 82 | 445 | 16.9 | 5483 | 13.0 | 0.209 |
| 10.119 | 8.7343 | 81 | 308 | 11.7 | 2978 | 7.0 | 0.164 |
| 11.734 | 7.5357 | 115 | 744 | 28.3 | 7725 | 18.3 | 0.176 |
| 12.639 | 6.9981 | 115 | 810 | 30.8 | 9343 | 22.1 | 0.196 |
| 13.280 | 6.6617 | 108 | 740 | 28.1 | 7329 | 17.3 | 0.168 |
| 14.676 | 6.0308 | 101 | 385 | 14.6 | 5677 | 13.4 | 0.251 |
| 15.196 | 5.8257 | 110 | 137 | 5.2 | 1380 | 3.3 | 0.171 |
| 15.959 | 5.5490 | 118 | 393 | 14.9 | 4367 | 10.3 | 0.189 |
| 16.838 | 5.2613 | 171 | 288 | 10.9 | 2160 | 5.1 | 0.128 |
| 17.442 | 5.0804 | 157 | 1216 | 46.2 | 14502 | 34.3 | 0.203 |
| 17.861 | 4.9621 | 184 | 347 | 13.2 | 4667 | 11.0 | 0.228 |
| 18.664 | 4.7505 | 195 | 164 | 6.2 | 1612 | 3.8 | 0.167 |
| 19.480 | 4.5533 | 188 | 981 | 37.3 | 14030 | 33.2 | 0.243 |
| 19.839 | 4.4716 | 183 | 2632 | 100.0 | 42278 | 100.0 | 0.273 |
| 20.321 | 4.3666 | 151 | 535 | 20.3 | 7766 | 18.4 | 0.247 |
| 21.420 | 4.1449 | 152 | 2459 | 93.4 | 24004 | 56.8 | 0.166 |
| 22.022 | 4.0330 | 148 | 45 | 1.7 | 547 | 1.3 | 0.205 |
| 22.236 | 3.9948 | 142 | 55 | 2.1 | 546 | 1.3 | 0.169 |
| 23.400 | 3.7985 | 182 | 339 | 12.9 | 2457 | 5.8 | 0.123 |
| 23.940 | 3.7141 | 128 | 560 | 21.3 | 11859 | 28.1 | 0.360 |
| 24.317 | 3.6573 | 154 | 241 | 9.2 | 3197 | 7.6 | 0.225 |
| 24.902 | 3.5727 | 119 | 139 | 5.3 | 991 | 2.3 | 0.121 |
| 25.983 | 3.4265 | 122 | 577 | 21.9 | 8147 | 19.3 | 0.240 |
| 26.358 | 3.3785 | 124 | 519 | 19.7 | 7535 | 17.8 | 0.247 |
| 27.218 | 3.2737 | 124 | 41 | 1.6 | 192 | 0.5 | 0.080 |
| 27.583 | 3.2313 | 138 | 52 | 2.0 | 283 | 0.7 | 0.092 |
| 27.959 | 3.1887 | 122 | 305 | 11.6 | 8559 | 20.2 | 0.476 |
| 28.218 | 3.1599 | 120 | 682 | 25.9 | 10745 | 25.4 | 0.268 |
| 28.980 | 3.0786 | 117 | 256 | 9.7 | 2153 | 5.1 | 0.143 |
| 29.782 | 2.9975 | 129 | 227 | 8.6 | 2593 | 6.1 | 0.194 |
| 30.141 | 2.9626 | 126 | 89 | 3.4 | 2182 | 5.2 | 0.416 |
| 30.521 | 2.9265 | 137 | 139 | 5.3 | 1989 | 4.7 | 0.243 |
| 31.337 | 2.8522 | 122 | 230 | 8.7 | 2809 | 6.6 | 0.208 |
| 31.921 | 2.8013 | 119 | 50 | 1.9 | 206 | 0.5 | 0.071 |
| 32.221 | 2.7760 | 128 | 81 | 3.1 | 1148 | 2.7 | 0.241 |
| 32.703 | 2.7361 | 106 | 271 | 10.3 | 4202 | 9.9 | 0.264 |
| 33.179 | 2.6979 | 138 | 115 | 4.4 | 1171 | 2.8 | 0.174 |
| 33.900 | 2.6422 | 120 | 145 | 5.5 | 1908 | 4.5 | 0.224 |
| 34.405 | 2.6046 | 135 | 103 | 3.9 | 602 | 1.4 | 0.100 |
| 35.317 | 2.5393 | 125 | 266 | 10.1 | 3059 | 7.2 | 0.196 |
| 35.820 | 2.5048 | 108 | 38 | 1.4 | 208 | 0.5 | 0.093 |
| 36.542 | 2.4570 | 102 | 85 | 3.2 | 750 | 1.8 | 0.150 |
| 36.922 | 2.4326 | 99 | 145 | 5.5 | 3173 | 7.5 | 0.373 |
| 38.078 | 2.3613 | 113 | 40 | 1.5 | 203 | 0.5 | 0.087 |
| 38.721 | 2.3236 | 103 | 132 | 5.0 | 3859 | 9.1 | 0.497 |
| 39.094 | 2.3023 | 108 | 427 | 16.2 | 4195 | 9.9 | 0.167 |

*FWHM = full width at half-maximum

Form I was also characterized by differential scanning calorimetry (DCS) using a Q100 modulated differential scanning calorimeter (TA Instruments; New Castle, Del.) at a scan rate of a 5° C. per minute (the instrument was calibrated using Indium). FIG. 2 presents the DSC thermogram for Form I. Crystalline Form I exhibited an endothermic transition with an onset of about 114°-118° C.

### Example 2

### Preparation and Characterization of Form I Utilizing Hot Crystallization

Ten grams of sufentanil citrate were dissolved in 50 mL of isopropanol at about 70-80° C. (i.e., the boiling temperature of isopropanol). The solution was cooled in an ice bath to induce crystallization. The resultant crystals were isolated and dried to obtain substantially pure crystalline Form I. The crystals were characterized by X-ray powder diffraction (see FIG. 1 and Table 1) and differential scanning calorimetry (see FIG. 2).

### Example 3

### Characterization of Form I in Slurry Preparation

To determine whether Form I changes its crystalline form over time, a slurry preparation was prepared. For this, sufentanil citrate was mixed with isopropanol in a small flask to form a saturated solution. Then, additional solid (Form I) sufentanil citrate was added to form a slurry. The resulting slurry was then stirred for a designated period of time using a magnetic stir-bar. The resulting crystals were collected, dried, and characterized by X-ray powder diffraction (see FIG. 1 and Table 1). It was found that the Form I crystals did not change.

### Example 4

### Preparation and Characterization of Form II Utilizing Hot Crystallization

A small volume of water was saturated with sufentanil citrate at an elevated temperature. The solution was then cooled in an ice bath to initiate crystal formation. The resultant crystals were isolated and dried to obtain substantially pure crystalline Form II.

The crystalline Form II was characterized by X-ray powder diffraction spectrometry as detailed above in Example 1. Table 2 summarizes the X-ray powder diffraction data, and FIG. 3 presents the characteristic X-ray powder diffraction pattern for the Form II crystalline form. This crystalline form exhibited characteristic peaks at about 5.6, about 11.4, about 19.1, about 20.5, about 21.2, and about 23.2 degrees 2-theta.

Form II was also characterized by DSC as detailed above in Example 1. FIG. 4 presents the DSC thermogram for Form II. This crystalline form exhibited a broad endothermic transition below about 75° C. and an endothermic transition with an onset of about 114-118° C.

TABLE 2
X-Ray Powder Diffraction Spectral Lines of Form II.

| | d Value | | Intensity | | | | |
|---|---|---|---|---|---|---|---|
| 2-Theta | (Å) | Background | Height | % | Intensity Area | % | FWHM* |
| 5.597 | 15.7779 | 100 | 1087 | 100.0 | 9815 | 100.0 | 0.154 |
| 7.569 | 11.6705 | 74 | 49 | 4.6 | 386 | 3.9 | 0.132 |
| 9.977 | 8.8586 | 60 | 201 | 18.5 | 2802 | 28.5 | 0.237 |
| 10.582 | 8.3532 | 66 | 131 | 12.1 | 1073 | 10.9 | 0.139 |
| 11.379 | 7.7699 | 63 | 224 | 20.7 | 3046 | 31.0 | 0.231 |
| 11.840 | 7.4683 | 54 | 155 | 14.3 | 1518 | 15.5 | 0.167 |
| 12.232 | 7.2302 | 58 | 30 | 2.8 | 170 | 1.7 | 0.097 |
| 13.419 | 6.5929 | 52 | 187 | 17.2 | 1928 | 19.6 | 0.175 |
| 13.994 | 6.3233 | 52 | 40 | 3.7 | 450 | 4.6 | 0.191 |
| 14.424 | 6.1358 | 50 | 81 | 7.5 | 1633 | 16.6 | 0.341 |
| 14.616 | 6.0556 | 48 | 61 | 5.6 | 1633 | 16.6 | 0.459 |
| 15.463 | 5.7259 | 52 | 61 | 5.6 | 824 | 8.4 | 0.229 |
| 15.698 | 5.6406 | 55 | 127 | 11.7 | 1445 | 14.7 | 0.193 |
| 16.242 | 5.4528 | 58 | 170 | 15.6 | 1673 | 17.0 | 0.168 |
| 16.704 | 5.3032 | 63 | 148 | 13.6 | 1853 | 18.9 | 0.213 |
| 17.395 | 5.0940 | 65 | 59 | 5.5 | 340 | 3.5 | 0.097 |
| 17.848 | 4.9657 | 62 | 63 | 5.8 | 456 | 4.6 | 0.124 |
| 18.268 | 4.8524 | 63 | 31 | 2.9 | 196 | 2.0 | 0.107 |
| 18.665 | 4.7502 | 64 | 64 | 5.9 | 751 | 7.7 | 0.199 |
| 19.058 | 4.6529 | 63 | 285 | 26.2 | 6611 | 67.4 | 0.395 |
| 19.459 | 4.5580 | 62 | 115 | 10.5 | 1887 | 19.2 | 0.280 |
| 20.280 | 4.3753 | 77 | 125 | 11.5 | 1442 | 14.7 | 0.197 |
| 20.522 | 4.3242 | 55 | 165 | 15.2 | 3679 | 37.5 | 0.379 |
| 21.179 | 4.1916 | 72 | 316 | 29.1 | 3184 | 32.4 | 0.171 |
| 22.581 | 3.9344 | 59 | 67 | 6.1 | 1132 | 11.5 | 0.289 |
| 22.917 | 3.8775 | 64 | 89 | 8.2 | 2542 | 25.9 | 0.486 |
| 23.160 | 3.8374 | 55 | 122 | 11.2 | 4025 | 41.0 | 0.562 |
| 23.637 | 3.7610 | 68 | 52 | 4.8 | 488 | 5.0 | 0.160 |
| 23.858 | 3.7267 | 63 | 141 | 13.0 | 1415 | 14.4 | 0.171 |
| 24.270 | 3.6644 | 53 | 34 | 3.1 | 655 | 6.7 | 0.326 |
| 24.496 | 3.6310 | 48 | 61 | 5.6 | 655 | 6.7 | 0.183 |
| 25.024 | 3.5556 | 42 | 33 | 3.1 | 448 | 4.6 | 0.229 |
| 25.374 | 3.5073 | 41 | 70 | 6.5 | 1053 | 10.7 | 0.254 |
| 25.843 | 3.4448 | 38 | 43 | 3.9 | 517 | 5.3 | 0.206 |
| 27.603 | 3.2289 | 53 | 102 | 9.4 | 1191 | 12.1 | 0.198 |
| 28.186 | 3.1635 | 56 | 45 | 4.2 | 853 | 8.7 | 0.321 |
| 28.542 | 3.1248 | 57 | 66 | 6.1 | 1408 | 14.3 | 0.360 |
| 29.051 | 3.0712 | 48 | 33 | 3.1 | 1039 | 10.6 | 0.532 |
| 30.196 | 2.9573 | 35 | 41 | 3.7 | 367 | 3.7 | 0.153 |
| 31.138 | 2.8699 | 39 | 38 | 3.5 | 594 | 6.1 | 0.268 |
| 31.683 | 2.8219 | 37 | 49 | 4.5 | 1227 | 12.5 | 0.423 |
| 32.872 | 2.7224 | 37 | 26 | 2.4 | 419 | 4.3 | 0.275 |
| 33.596 | 2.6654 | 43 | 34 | 3.1 | 484 | 4.9 | 0.245 |
| 35.059 | 2.5574 | 38 | 35 | 3.3 | 450 | 4.6 | 0.216 |
| 36.056 | 2.4890 | 38 | 24 | 2.2 | 181 | 1.8 | 0.129 |
| 38.381 | 2.3434 | 35 | 47 | 4.3 | 794 | 8.1 | 0.289 |
| 38.810 | 2.3185 | 31 | 23 | 2.1 | 443 | 4.5 | 0.331 |

*FWHM = full width at half-maximum

What is claimed is:

1. A crystalline form of sufentanil citrate, N-[4-(methoxymethyl)-1-[2-(2-thienyl)ethyl]4-piperidinyl]-N-phenylpropanamide, 2-hydroxy-1,2,3-propanetricarboxylate, wherein the crystalline form is hydrous Form II.

2. The crystalline form of claim 1, wherein the crystalline form exhibits an X-ray powder diffraction pattern having characteristic peaks expressed in degrees 2-theta at about 5.6, about 10.0, about 11.4, about 13.4, about 19.1, and about 21.2.

3. The crystalline form of claim 1, wherein the crystalline form exhibits an endothermic transition with an onset of about 114°-118° C. as measured by differential scanning calorimetry.

4. The crystalline form of claim 1, wherein the crystalline form comprises no more than about 5% by weight of crystalline Form I.

5. A pharmaceutical composition, comprising:
a) Form II crystalline form of sufentanil citrate, N-[4-(methoxymethyl)-1-[2-(2-thienyl)ethyl]4-piperidinyl]-N-phenylpropanamide, 2-hydroxy-1,2,3-propanetricarboxylate; and
b) a pharmaceutically acceptable excipient.

6. The pharmaceutical composition of claim 5, wherein the crystalline Form II exhibits an X-ray powder diffraction pattern having characteristic peaks expressed in degrees 2-theta at about 5.6, about 10.0, about 11.4, about 13.4, about 19.1, and about 21.2.

7. The pharmaceutical composition of claim 5, wherein the crystalline Form II exhibits an endothermic transition with an onset of about 114°-118° C. as measured by differential scanning calorimetry.

8. The pharmaceutical composition of claim 5, wherein the crystalline form comprises no more than about 5% by weight of crystalline Form I.

9. A process for preparing a substantially pure anhydrous crystalline Form I of sufentanil citrate, N-[4-(methoxymethyl)-1-[2-(2-thienyl)ethyl]-4-piperidinyl]-N-phenylpropanamide, 2-hydroxy-1,2,3-propanetricarboxylate, the process comprising:
a) contacting sufentanil citrate, N-[4-(methoxymethyl)-1-[2-(2-thienyl)ethyl]-4-piperidinyl]-N-phenylpropanamide, 2-hydroxy-1,2,3-propanetricarboxylate, with a solvent to form a saturated or a near saturated solution; and
b) forming crystals of substantially pure anhydrous crystalline Form I.

10. The process of claim 9, wherein the crystals are formed by slow evaporation of the solvent.

11. The process of claim 9, wherein the saturated or near saturated solution is heated to about the boiling point of the solvent during step (a).

12. The process of claim 11, wherein the crystals are formed by cooling the solution.

13. The process of claim 9, further comprising the step of collecting the crystals of substantially pure crystalline Form I.

14. The process of claim 13, further comprising the step of drying the crystals of substantially pure crystalline Form I.

15. The process of claim 9, wherein the solvent is selected from the group consisting of a protic solvent selected from the group consisting of methanol, ethanol, isopropanol, n-propanol, isobutanol, n-butanol, s-butanol, t-butanol, formic acid, and acetic acid; an aprotic solvent selected from the group consisting of acetone, acetonitrile, dichloromethane, and tetrahydrofuran; and combinations thereof.

16. The process of claim 15, wherein the solvent is an alcohol selected from the group consisting of methanol, ethanol, isopropanol, n-propanol, isobutanol, n-butanol, s-butanol, t-butanol, and combinations thereof.

17. The process of claim 16, wherein the solvent is ethanol or isopropanol.

18. The process of claim 9, wherein the crystalline Form I exhibits an X-ray powder diffraction pattern having characteristic peaks expressed in degrees 2-theta at about 11.7, about 12.6, about 13.3, about 17.4, about 19.5, about 19.8, and about 21.4.

19. The process of claim 9, wherein the crystalline Form I exhibits an endothermic transition with an onset of about 136°-138° C. as measured by differential scanning calorimetry.

20. A process for preparing a substantially pure hydrous crystalline Form II of sufentanil citrate, N-[4-(methoxymethyl)-1-[2-(2-thienyl)ethyl]-4-piperidinyl]-N-phenylpropanamide, 2-hydroxy-1,2,3-propanetricarboxylate, the process comprising:
a) contacting sufentanil citrate, N-[4-(methoxymethyl)-1-[2-(2-thienyl)ethyl]-4-piperidinyl]-N-phenylpropanamide, 2-hydroxy-1,2,3-propanetricarboxylate, with a solvent to form a saturated or a near saturated solution; and
b) forming crystals of substantially pure hydrous crystalline Form II.

21. The process of claim 20, wherein the saturated or near saturated solution is heated to about the boiling point of the solvent during step (a).

22. The process of claim 21, wherein the crystals are formed by cooling the solution.

23. The process of claim 20, further comprising the step of collecting the crystals of substantially pure crystalline Form I.

24. The process of claim 23, further comprising the step of drying the crystals of substantially pure crystalline Form I.

25. The process of claim 20, wherein the solvent is selected from the group consisting of a protic solvent selected from the group consisting of water, methanol, ethanol, isopropanol, n-propanol, isobutanol, n-butanol, s-butanol, t-butanol, formic acid, and acetic acid; an aprotic solvent selected from the group consisting of acetone, acetonitrile, dichloromethane, and tetrahydrofuran; and combinations thereof.

26. The process of claim 25, wherein the solvent is water.

27. The process of claim 20, wherein the crystalline Form II exhibits an X-ray powder diffraction pattern having characteristic peaks expressed in degrees 2-theta at about 5.6, about 10.0, about 11.4, about 13.4, about 19.1, and about 21.2.

28. The process of claim 20, wherein the crystalline Form II exhibits an endothermic transition with an onset of about 114°-118° C. as measured by differential scanning calorimetry.
